(19) United States
(12) Patent Application Publication
(10) Pub. No.: US 2004/0111143 A1
(43) Pub. Date: Jun. 10, 2004

(54) Introducer sheath for the ostial placement of a stent

(76) Inventors: Robert E. Fischell, Dayton, MD (US); Tim A. Fischell, Kalamazoo, MI (US)

Correspondence Address: Robert E. Fischell, 14600 Viburnum Drive, Dayton, MD, 21036, US

(21) Appl. No.: 10/310,209

(22) Filed: Dec. 6, 2002

(51) Int. Cl. A61F 2/06

(52) U.S. Cl. 623/1.11

(57) ABSTRACT

Disclosed is a catheter that has the form of an introducer sheath having an expandable radiopaque flange situated at the sheath's distal end. The distal end of the flange is adapted to be placed at the ostial plane of a stenosed artery. Also disclosed is a method for accurately placing a stent at the ostium of an artery that would have an ostial stenosis. Examples of such arteries that have ostial stenoses are the right and left main coronary arteries, a saphenous vein graft as used in coronary bypass surgery and the renal arteries. The sheath is placed within a guiding catheter that has its distal end located close to the ostium of an artery where there is a stensosis. When the introducer sheath is advanced beyond the distal end of the guiding catheter, a flange expands at the end of the sheath. The flange is then pushed forward until its distal end is at the ostial plane of the ostium of the stenosed artery. A stent on a stent delivery system can then be positioned accurately relative to the expanded flange.

### FIELD OF USE

This invention is in the field of catheter devices for placing stents within an arterial stenosis.

### BACKGROUND OF THE INVENTION

Although most stenoses do not occur at the ostium of an artery, there are thousands of cases each month where the mouth of an artery (the ostium) is substantially obstructed at its aortic take-off. In such cases, the interventional cardiologist or radiologist is frequently unable to place the stent accurately with its proximal end located exactly at the ostial plane. Two types of incorrect stent positions are (1) when the stent's proximal end extends more than 2 mm into the aorta, and (2) when the stent's proximal end is placed more than 2 mm into the artery distal to the ostial plane.

### SUMMARY OF THE INVENTION

One aspect of the present invention is a catheter that has the form of a sheath having an expandable flange situated at the sheath's distal end. A second aspect of the present invention is a method for accurately placing a stent at the ostium of an artery that would have an ostial stenosis. Examples of such arteries that have ostial stenoses are the right and left main coronary arteries, a saphenous vein graft as used in coronary bypass surgery and the renal arteries.

The method for using this invention would be to first place a guiding catheter through the aorta in a conventional manner so that its distal end will be engaged within or near the ostium of the artery that is to be stented. A guide wire would then be advanced through the guiding catheter until its distal end was placed distal to the stenosis. If pre-dilitation of the ostial stenosis was needed, a balloon angioplasty catheter would be advanced over the guide wire and through the guiding catheter and the catheter's balloon would be inflated to pre-dilate the stenosis. After the balloon angioplasty catheter was removed from the guiding catheter (or if no pre-dilatation was required) then the ostial placement introducer sheath and a stent delivery system within the introducer sheath would be advanced over the guide wire and through the guiding catheter. The sheath would be positioned with its distal end placed a few centimeters proximal to the distal end of the guiding catheter. The stent delivery system would have its proximal radiopaque marker band placed just beyond the distal end of the ostial stenosis. While retaining the guide wire and a distal portion of the stent delivery system in the artery, the guiding catheter with the sheath inside would then be pulled a short distance back into the aorta. The sheath would then be advanced until its expandable flange at the sheath's distal end extended beyond the guiding catheter's distal end, thus allowing the expandable flange to fully expand. The guiding catheter would then be advanced over the sheath until the guiding catheter's distal end pushed the distal end of the sheath's expandable flange against the wall of the aorta and generally aligned with the ostium of the artery that is to be stented. The plane of the distal end of the expandable flange would then be situated at the artery's ostial plane. Since the expandable flange would be formed from a plastic that included a radiopaque substance or from a metal that is radiopaque, or a combination of the two, the interventional cardiologist who is performing this procedure would have a clear marker of the ostial plane of the artery that is to have a stent placed at the ostial stenosis of that artery. The interventional cardiologist would then pull the stent delivery system back until the proximal radiopaque marker band within the balloon of the stent delivery system was aligned with the expandable flange. The balloon would then be inflated to deliver the stent accurately into the ostial stenosis with the stent's proximal end lying within 2 mm of the ostial plane of the artery. It is expected that an experienced interventional cardiologist could place the proximal end of the stent within one to one-half a millimeter from the ostial plane.

The main object of this invention is to provide a means and method for accurately placing the proximal end of a stent within ±2 mm of the ostial plane of an artery that has a stenosis located at or near the ostium of that artery.

Another object of this invention is to place the proximal end of a stent within ±1.0 mm of the ostial plane of an artery that has a stenosis located at or near the artery's ostium.

These and other objects and advantages of this invention will become obvious to a person of ordinary skill in this art upon reading the detailed description of this invention including the associated drawings as presented herein.

### BRIEF DESCRIPTION OF THE DRAWINGS

FIG. 1 is a side view of a catheter system for placing the proximal end of a stent in close proximity to the ostial plane of an artery that has an ostial stenosis.

FIG. 2 is a longitudinal cross section of a tear-away insertion slide for inserting the distal end of the ostial positioning sheath into Touhy-Borst fitting and the guiding catheter.

FIG. 3 is a longitudinal cross section of a distal portion of a guiding catheter into which the sheath with its expandable flange has been placed; the expandable flange being shown in its folded state.

FIG. 4 is a side view of a distal portion of a guiding catheter with the expandable flange of the sheath shown in its expanded position.

FIG. 5 is an end view of the expandable flange.

FIG. 6 is a cross section of the guiding catheter and sheath showing the distal end plane of the expandable flange placed at the ostial plane of an artery having an ostial stenosis.

### DETAILED DESCRIPTION OF THE INVENTION

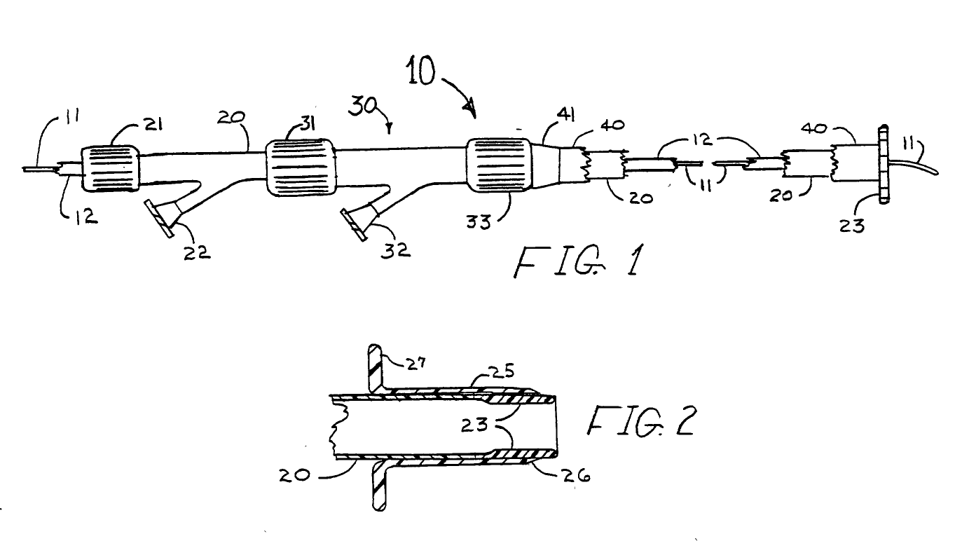

FIG. 1 is a side view of a catheter system 10 whose object is to accurately place a stent with its proximal end being placed close to the ostial plane of an artery having an ostial stenosis. The catheter system 10 would include a guide wire 11, a stent delivery system 12, an ostial positioning introducer sheath 20 having an expandable distal end flange 23, a Touhy-Borst fitting 30 and a guiding catheter 40. At its proximal end, the sheath 20 would have an adjustable seal fitting 21 and a Luer fitting 22 on a side arm of the sheath 20. The adjustable seal fitting 21 would initially be somewhat loosened to allow the stent delivery system 12 to be advanced or pulled back relative to the sheath 20 while preventing the free release of arterial blood. The adjustable seal fitting 21 would be tightened prior to delivering the stent into an arterial stenosis. The Luer fitting 22 would be used for access to the lumen of the sheath 20 for flushing the lumen with saline solution or for introducing contrast medium for visualizing certain aspects of the vascular geometry.

The Touhy-Borst adaptor 30 has an adjustable seal fitting 31 that can initially be slightly loosened to allow the sheath 20 to be advanced or pulled back through the guiding catheter 40 without excessive blood leakage. When the expandable flange 23 is in its correct position for placement at the ostial plane, (as seen in FIGS. 1, 4 and 6) the adjustable seal fitting 31 can be tightened to hold a fixed position of the flange 23 relative to the guiding catheter 40 during stent deployment. The Luer fitting 32, being in fluid communication with the lumen of the guiding catheter 40, can be used for flushing the lumen with saline solution and/or for injecting contrast medium. The Luer connector 33 is used to form a removable fluidic seal with the Luer fitting 41 of the guiding catheter 40.

FIG. 2 is a longitudinal cross section of a tear-away insertion slide 25 that has a tapered distal portion 26 and a flange 27. In FIG. 2, the slide 25 is shown with its distal portion 26 covering the petals of the expandable flange 23 to keep them retracted for easy insertion into the proximal end of the Touhy-Borst fitting 30. After the distal portion 26 is placed into the Touhy-Borst fitting 30, the flange 27 is used to pull the slide 25 in a proximal direction to remove the slide 25 from the Touhy-Borst fitting 30 while retaining the expandable flange 23 with its petals retracted within the Touhy-Borst fitting 30. The slide 25 can then be torn off the sheath 20 along a tear line which is designed into the slide 25 for easy tearing without the use of excessive force. It is also conceived that the slide 25 could be pushed to and be retained near the proximal end of the sheath 20.

FIG. 3 is a cross section of distal portions of the guiding catheter 40 and the sheath 20. The guiding catheter 40 has a radiopaque end section 42. The sheath 20, having a lumen 24, is shown with its expandable flange 23 in its unexpanded state within the guiding catheter 40. In this state, the guiding catheter 40 can be advanced through an introducer sheath at the patient's groin until its distal end is within the ostium of the artery that is to be stented.

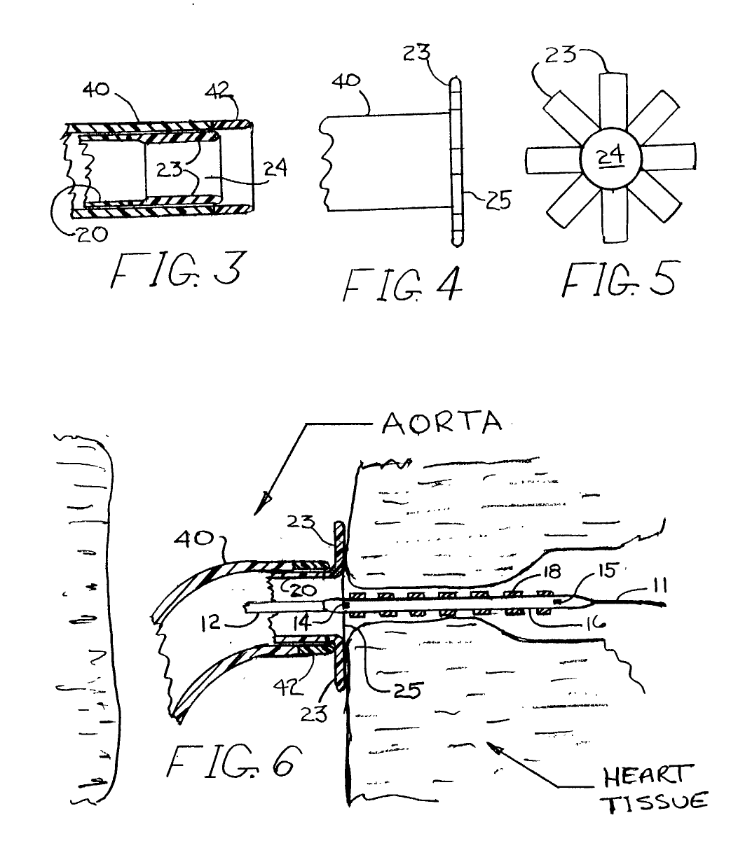

FIG. 4 is a side view of a distal portion of the guiding catheter 40 showing the expandable flange 23 in its expanded position. The distal plane 25 of the expandable flange 23 is optimally placed at the ostial plane of the artery that is to be stented as shown in FIG. 6. FIG. 5 is an end view of the expandable flange 23 which also shows the lumen 24 of the sheath 20. FIG. 5 shows the expandable flange 23 in the form of a flower-like arrangement with multiple petals. Although 8 petals are shown in FIG. 5, as few as 2 or as many as 16 petals could be used for an effective expandable flange.

FIG. 6 is a cross section of a distal portion of the catheter system 10 shown with the distal plane 25 of the expandable flange 23 placed at the ostial plane of a stenosed artery. Any such placement can be defined as having the distal plane 25 “co-planar” with the ostial plane. FIG. 6 also shows the guide wire 11 and the stent delivery system 12 which has a proximal radiopaque marker band 14, a distal radiopaque marker band 15 and a stent 18 mounted onto a balloon 16.

At the start of the stenting procedure, the catheter system 10 would be positioned as shown in FIG. 1, except that the expandable flange 23 would be placed inside the guiding catheter 40 as shown in FIG. 3. The catheter system 10 and the guide wire 11 could then be advanced through a conventional introducer sheath (not shown) typically placed at the groin of the patient into whom the stent 18 is to be placed. The guide wire 11 would be placed into and through the ostial stenosis and the guiding catheter 40 would be advanced until it was placed through the arterial ostium. The guiding catheter 40 would then be pulled back into the aorta. The sheath 20 would then be advanced through the guiding catheter 40 until the expandable flange 23 extended out of the distal end of the guiding catheter 40. The guiding catheter 40 would then be pushed forward in a distal direction so as to obtain the configuration as shown in FIG. 6.

With the configuration as shown in FIG. 6, the interventional cardiologist would be able to clearly visualize the distal plane 25 of the expandable flange 23 and also visualize the proximal radiopaque marker band 14. When the radiopaque marker band 14 is pulled backward inside the expandable flange 23 so that it just disappears, then the proximal end of the stent 18 would be placed exactly at the plane of the ostium of the vessel which is also the distal plane 25 of the expandable flange 23. The balloon 16 would then be inflated to deliver the stent 18 into the ostial stenosis. Thus, the interventional cardiologist who is implanting the stent 18 should be able to readily place the proximal end of the stent 18 within ±2 mm of the ostial plane. With some experience, it is expected that the stent 18 could be placed within at least ±1.0 mm of the ostial plane and probably within ±0.5 mm.

Although one method for accurately placing the stent 18 into an ostial stenosis has been described herein, it should be understood that there are several other ways that the present invention can be used to provide accurate stent positioning within an ostial stenosis. For example the guiding catheter 40 could first be placed over a 0.035 inch diameter guide wire and into the lumen of the ostial stenosis. That larger diameter guide wire could then be removed and a 0.014 inch diameter guide wire could be placed through the stenosis. The stent delivery system 12 could then be advanced over that guide wire and positioned as shown in FIG. 5. The expandable flange 23 could then be deployed as described herein.

Instead of using the expandable flange 23 as described herein, a sheath with an expandable balloon mounted on a distal portion of the sheath 20 could also be used to reference the ostial plane. It is further anticipated that the guiding catheter 40 could have a means at its distal end for referencing the ostial plane. This could be done, for example, by having a balloon at the distal end of the guiding catheter 40 that is inflated so as to have a distal plane that could be placed so as to be co-planar with the ostial plane. Furthermore, the guiding catheter could have an expandable flange at its distal end according to the general design concept presented herein.

Various other modifications, adaptations and alternative designs are of course possible in light of the teachings as presented herein. Therefore it should be understood that, while still remaining within the scope and meaning of the appended claims, this invention could be practiced in a manner other than that which is specifically described herein.

What is claimed is:

1. An introducer sheath for facilitating the placement of the proximal end of a stent within ±2 mm of the ostial plane of an artery that has an ostial stenosis, the sheath having an expandable distal portion that has a distal plane, the sheath being adapted to be placed within a guiding catheter and being further adapted for placement of its distal plane so as to be substantially co-planar with the ostial plane of the artery when the guiding catheter is urged forward in a distal direction.

2. The sheath of claim 1 wherein the expandable distal portion of the sheath is generally radiopaque.

3. The sheath of claim 1 wherein the expandable distal portion of the sheath is in the form of an expandable flange.

4. The sheath of claim 3 wherein the expandable flange is formed from a radiopaque plastic material.

5. The sheath of claim 3 wherein the expandable flange is formed from a radiopaque metal.

6. The sheath of claim 3 wherein the expandable flange is formed from a combination of a plastic material and a metal, the combination being generally radiopaque.

7. The sheath of claim 1 wherein the expandable flange is generally in the shape of a flower that has at least two petals.

8. The sheath of claim 1 wherein the expandable flange has at least four petals.

9. The sheath of claim 1 wherein the expandable distal portion has an outer diameter of less than 10 mm.

10. A method for placing the proximal end of a stent within an artery that has an ostial stenosis so that the stent's proximal end is positioned within ±2 mm of the artery's ostial plane, the method including the following steps:
a) advancing an introducer sheath that has an expandable distal portion within a guiding catheter with the expandable distal portion in its unexpanded state;
b) causing the expandable distal portion to expand in a region beyond the distal end of the guiding catheter to form an expanded flange;
c) urging the guiding catheter in a forward, distal direction so that a distal plane of the expanded flange is placed substantially co-planar with the ostial plane of the artery that has the ostial stenosis; and
d) positioning a stent within the stenosis of the artery so that the stent's proximal radiopaque marker band is situated relative to the distal plane of the expanded flange of the sheath so that the stent's proximal end is located within ±2 mm of the ostial plane.

11. The method of claim 10 wherein the sheath's expanded flange is generally shaped in the form of a flower with at least two petals.

12. The method of claim 10 wherein the expanded flange is generally radiopaque.

13. The method of claim 10 wherein the expandable distal portion is in the form of an expandable balloon that is adapted to be inflated so as to form a distal plane that can be placed essentially co-planar with the ostial plane of the artery.
